(19) United States
(12) Patent Application Publication
(10) Pub. No.: US 2021/0407647 A1
(43) Pub. Date: Dec. 30, 2021

(54) SYSTEM AND METHOD FOR PERSONALIZED TREATMENT WITH ARTIFICIAL INTELLIGENCE

(71) Applicant: KONINKLIJKE PHILIPS N.V., Eindhoven (NL)

(72) Inventors: Yash Parag MOKASHI, Pittsburgh, PA (US); Akhil CHATURVEDI, Pittsburgh, PA (US); Mantek CHADHA, Pittsburgh, PA (US)

(21) Appl. No.: 17/357,449

(22) Filed: Jun. 24, 2021

Related U.S. Application Data
(60) Provisional application No. 63/046,354, filed on Jun. 30, 2020.

(51) Int. Cl. G16H 20/30; G06N 20/00; G16H 10/60; G16H 10/20; G16H 40/63; G16H 20/60

(52) U.S. Cl. CPC G16H 20/30; G06N 20/00; G16H 20/60; G16H 10/20; G16H 40/63; G16H 10/60

(57) ABSTRACT

A method of providing personalized treatment includes gathering first information (402,404) associated with a user (400), determining a first user profile vector (500) based on first information associated with the user, determining a first treatment plan to move the user from the first user profile vector toward a target user profile vector (506), and providing the first treatment plan to the user.

### CROSS-REFERENCE TO PRIOR APPLICATION

This application claims the benefit of U.S. Provisional Application No. 63/046,354, filed on 30 Jun. 2020. This application is hereby incorporated by reference herein.

### BACKGROUND OF THE INVENTION

### 1. Field of the Invention

The disclosed concept relates generally to devices and methods for determining and providing personalized treatment for users with artificial intelligence techniques.

### 2. Description of the Related Art

Habits and activities affect people in numerous ways. Many people would like to lead healthier lifestyles and general advice for leading a healthier lifestyle is often available. However, such general advice is not most effective for a particular person. For example, advising someone to eat healthier, sleep more, and get more exercise, while good advice, may not lead to the particular person following this advice. Moreover, for a person with a more particular goal other than generally living a more healthy lifestyle, this general type of advice is not optimal and does not take into account the person's particular situation and goal.

One example of something that many people would like to improve is sleep quality. Various habits and activities affect sleep quality. Through media and education, people are becoming more aware of such habits an activities. However, just telling a person to change their behavior is generally not sufficient. Behavior change is a more complex process. Telling people to stop their behaviors to improve sleep quality cold-turkey is not most effective. Behavior change in people could be more effective with intermediate steps and ongoing guidance through the process.

Current digital platforms that give sleep hygiene and habit recommendations inform users on what they should change in their habit and some even provide a plan on how to change these. However, these plans are generalized to user groups based on basic demographic and current habit information. They target a user-type based on heuristics and prior knowledge. This is an inefficient solution as it assumes one-size-fits all model. Even when taking into account personality types, these types of solutions are not optimal. In the domain of psychology, personality-types are used to club different people as having some common features. These personality-types can be used to treat or interact with a person in a way targeted to that personality type. However, the end result is still that user are generalized into groups.

As an alternative to digital platforms, personal trainers, coaches or therapists are found to be more effective in helping with behavior changes, when compared to generalized plans or even group coaching. This is because they can take the time to first understand the specific individual and devise a plan that is personalized to the individual, and modify the plan over time based on progress. This plan may be based on multiple factors, which might include the user's current behavioral, demographic, physical and emotional attributes, goals and reception to the coaching instructions. These are often not covered by the basic on-boarding surveys conducted by digital coaching platforms before recommending a plan. However, even with personal coaching, the effectiveness is still limited by the particular personal coach's knowledge.

Accordingly, a need exists for improving the development of personal treatment plans for users.

### SUMMARY OF THE INVENTION

Accordingly, it is an object of the disclosed concept to provide a device and method that develops and provides a personalized treatment plan to assist a user in achieving their desired goal.

As one aspect of the disclosed concept, a method of providing personalized treatment comprises: gathering first information associated with a user; determining a first user profile vector based on first information associated with the user; determining a first treatment plan to move the user from the first user profile vector toward a target user profile vector; and providing the first treatment plan to the user.

As one aspect of the disclosed concept, a system comprises: a user device structured to gather first information associated with a user; a backend system structured to receive the first information, to determine a first user profile vector based on first information associated with the user, to determine a first treatment plan to move the user from the first user profile vector toward a target user profile vector, and to provide the first treatment plan to the user device, wherein the user device is structured to provide the first treatment plan to the user.

As one aspect of the disclosed concept, a non-transitory computer readable medium having computer readable code thereon which, when executed by a processor causes the processor to implement a method of providing personalized treatment is provided. The method comprises: gathering first information associated with a user; determining a first user profile vector based on first information associated with the user; determining a first treatment plan to move the user from the first user profile vector toward a target user profile vector; and providing the first treatment plan to the user.

These and other objects, features, and characteristics of the disclosed concept, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIG. 1 is a flowchart of a method of developing and providing a personalized treatment plan to a user in accordance with an embodiment of the disclosed concept;

FIG. 2 is a flowchart of a method of determining a target user profile vector in accordance with an embodiment of the disclosed concept;

FIG. 3 is a flowchart of a method of selecting a treatment plan in accordance with an embodiment of the disclosed concept;

FIG. 4 is a conceptual diagram of developing a user profile vector in accordance with an embodiment of the disclosed concept;

FIG. 5 is a conceptual diagram of a user moving from an initial user profile vector to a target profile vector in accordance with an embodiment of the disclosed concept;

FIG. 6 is a schematic diagram of a system for developing and providing personalized treatment plans in accordance with an embodiment of the disclosed concept.

### DETAILED DESCRIPTION OF EXEMPLARY EMBODIMENTS

As required, detailed embodiments of the disclosed concept are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the disclosed concept in virtually any appropriately detailed structure.

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs.

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

In accordance with an embodiment of the disclosed concept, objective and subjective information associated with as user as well as information associated with a user's goals are gathered. Objective information is considered to be signals collected from a user directly through contact and non-contact sensors (e.g., without limitation, health tracking wearables for activity data, biosignals data, geolocation, facial data, voice signals (to determine mood, alertness, etc.), sleep data, height, weight, etc. collected from digital connected devices). Subjective information is considered to be any information derived from a user through questions that the user directly answers, so they are subjectively entered by the user through direct digital input questions (e.g., without limitation, yes/no, rating, scale, multiple choice, etc. type questions or open-ended text-entry/free-text questions). A user profile vector is determined for the user based on the objective and subjective information. A target user profile vector is also determined. The user profile vector is a mathematical representation of the state of the user at a particular point in time. The target user profile vector is a mathematical representation of the desired state of the user based on their goal. In accordance with an embodiment of the disclosed concept, a treatment plan is determined to move the user from their initial user profile vector towards the target user profile vector along an optimal path. The process may be iterative. For example, the user may move from the initial user profile vector to an intermediate user profile vector at which their treatment plan is reevaluated. The determination of the treatment plan may be determined, for example, with reinforced machine learning. In accordance with an embodiment of the disclosed concept, the coaching and/or treatment for the user is mathematically driven and personalized, resulting in the treatment plan being more effective in assisting the user to reach their goal. In an embodiment, the disclosed concept is applied to improving user sleep and respiratory health. However, it will be understood that the disclosed concept is applicable to a wide variety of coaching and/or treatment applications. Some embodiments of the disclosed concept will be described in more detail herein.

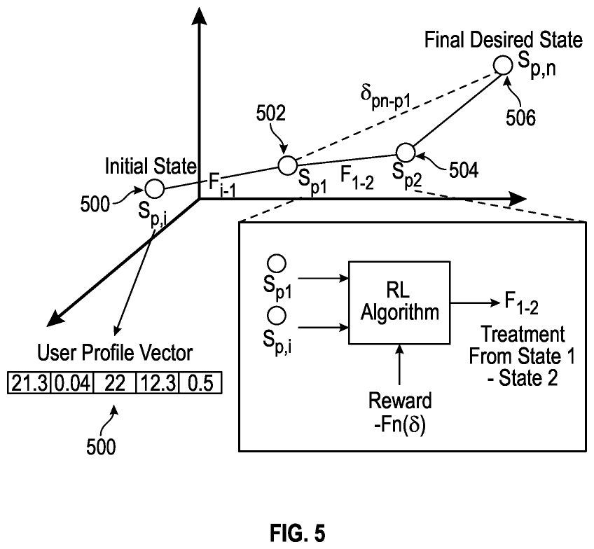

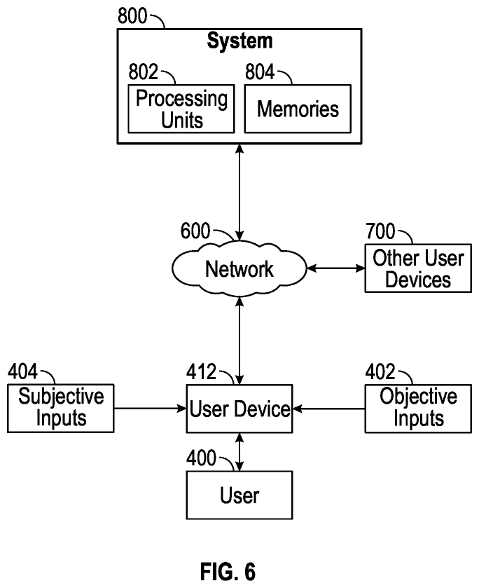

FIG. 5 is a conceptual diagram of a user moving from an initial user profile vector to a target profile vector in accordance with an embodiment of the disclosed concept. In FIG. 5, the user's progress over the time of the treatment plan is depicted. FIG. 6 is a schematic diagram of a system for developing and providing personalized treatment plans in accordance with an embodiment of the disclosed concept. The conceptual diagrams in FIGS. 5 and 6 will occasionally be referred to throughout the description to provide a clearer explanation of aspects of the disclosed concept. The disclosed concept will generally be described with respect to providing coaching and/or treatment related to sleep quality. However, as described above, the disclosed concept is applicable to many other fields as well.

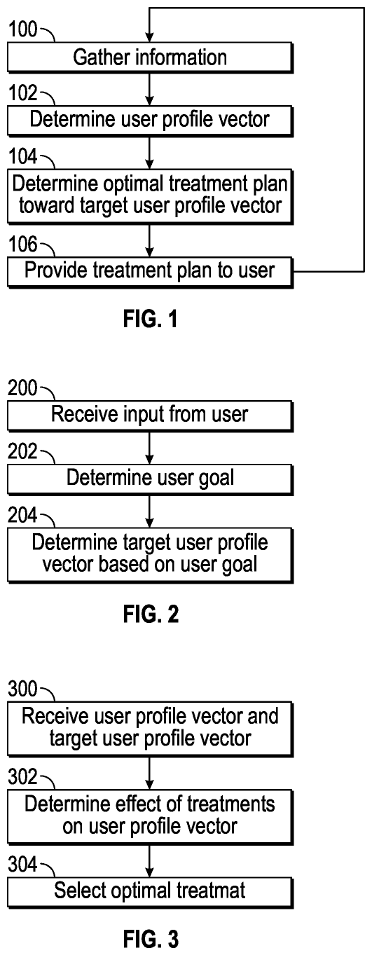

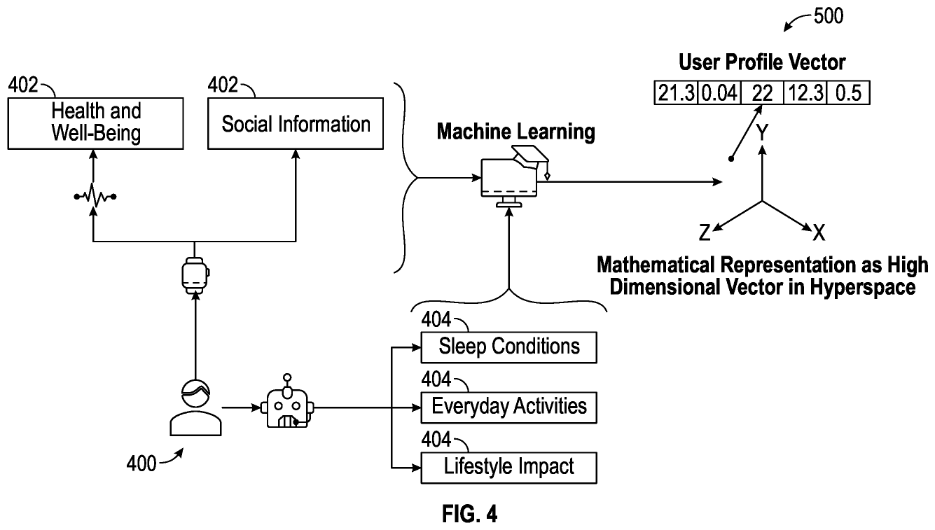

FIG. 1 is a flowchart of a method of developing and providing a personalized treatment plan to a user in accordance with an embodiment of the disclosed concept. The method begins at 100 where information associated with a user 400 (shown in FIG. 4) is gathered. The information may include objective information 402 and subjective information 404 associated with user 400. In an embodiment, the types of information gathered are relevant to a user goal. For example, in the case of sleep quality, the gathered information may include demographic information such as age and gender. Age affects the recommended sleep duration, the sleep requirement, ability to sleep, and sleeping patterns. Gender affects the sleep requirement and sleep problems. The gathered information may also include emotional attributes, which can affect sleep and the user's attitude toward behavior change.

Further types of information that may be gathered are caffeine intake. The time and amount of caffeine affects sleep. Additionally, other known and unknown factors like genetics affect how caffeine affects a user's sleep. Exercise activity can also be gathered, as the timing and type of exercise affects sleep. Stress levels may also be gathered, as these also affect sleep. The user's diet is also information that may be gathered, as it can also affect sleep. Furthermore, the information on the user's individual routine may be gathered, as a user's evening activity, such a use of electronic devices before bed, can affect sleep. While these are examples of types of information that may be gathered, it will be understood that the disclosed concept is not limited thereto. Other types of information may be gathered without departing from the scope of the disclosed concept.

Some additional types of information that may be gathered are subjective answers to direct questions to the user, such as their experience, how they are feeling, etc. Some other objective information that may be gathered is mood detection, alertness detection from speech, facial recognition, etc. It will also be appreciated that in applications other than sleep quality, different types of information may be gathered without departing from the scope of the disclosed concept.

The information may be gathered using various different methods. For example, user input may be one method. For example, the user or someone else may provide information, for example, in response to a questionnaire. Information, such as heart rate, respiratory signal data, sleep data, and exercise data, may be gathered from wearable devices such as smart watches or other smart devices. The user's social media interaction activity or other user activity through third party applications may also be sources of gathered information. Additionally, information may be gathered from user devices such as a user's smartphone or television could be gathered. Such information can be indicative of the user's location at certain times, the user's electronic device usage at certain times, or of the user's habits and other things that could be affecting their sleep. This type of information can also affect the type of treatment plan that will be most effective for the user.

Furthermore, information may be gathered based on natural language processing of the user. For example, a user may be prompted by an interactive device and the user's answers may be processed. Such information can be used to determine a user's psychological or emotional state. Such information may also be used to determine the user's goal. While various information sources have been described, it will be appreciated that the disclosed concept is not limited to these types of information sources and that the disclosed concept need not use all of these types of information sources. The information sources may be determined based on various factors such as their applicability, availability, and the user's consent in providing information or making the information source available.

Once information associated with user 400 is gathered, the method proceeds to 102. At 102, a user profile vector 500 is determined. User profile vector 500 is a mathematical representation of user 400 at a particular point in time. User profile vector 500 is based on information relevant to the type of coaching and/or treatment for user 400. For example, in the case of sleep coaching and/or treatment, user profile vector 500 is based on information relevant to user's 400 sleep.

In an embodiment, multimodal machine learning and knowledge mapping techniques are used to fuse data from various different input sources to create a high dimensional numeric representation of user 400 at a particular point in time. For example, the different types of information may be weighted based on their effect on user's 400 sleep. User profile vector 500 may include multiple variable, each comprised of one or more weighted or unweighted pieces of information. For example, user profile vector 500 (shown in FIG. 4) includes five variables which, when user profile vector 500 is determined, each have a numerical value.

User profile vector 500 provides a mathematical representation of user 400 at a particular point in time, which can be used to compare user 400 to other users and identify areas in which user 400 can change to meet their goal.

In an embodiment of the disclosed concept, a target user profile vector 506 (shown in FIG. 5) is also developed. Turning for the time to FIG. 2, FIG. 2 is a flowchart of a method of determining a target user profile vector in accordance with an embodiment of the disclosed concept. The method of FIG. 2 begins at 200 with receiving input associated with user's 400 goal from user 400. The input may be received from one or more of the various information sources previously described. At 202, the user's 400 goal is determined. In some cases, user's 400 goal may be explicitly provided, but in others it may be determined through the use of natural language processing or other techniques. User's 400 goal may be straightforward, such as getting eight hours of sleep a night, or it may be more subjective, such as having more energy during the day.

Once user's 400 goal is determined, the method proceeds to 204, where target user profile vector 506 is determined. Target user profile vector 506 may be determined by referencing a database containing user profile vectors from other users that, for example, may be similar to user 400 in other aspects, but have already met user's 400 goal. In this respect, the disclosed concept may have a pre-deployment phase, where data is collected on users and user profile vectors are determined for users through coaching and/or treatment. Once enough data is collected to effectively use machine learning on the data, the disclosed concept can move to a post-deployment phase where it can be applied to general users. It should be appreciated though, that in the post-deployment phase, data can continually be gathered into one or more databases. As more data is gathered, the machine learning techniques used to develop user profile vectors, target user profile vectors, and treatment and/or coaching plans can continually improve. As those skilled in the art will understand, machine learning recognizes patterns in data and its results can continually improve with more data. For example, the effect of information about the user on the relevant goal, sleep, for example, may be better determined as more data is gathered. Similarly, the effect of treatments on sleep or other goals, may be better determined.

Turning back to FIG. 1, after determining user profile vector 500 at step 102, the method proceeds to 104, where the optimal treatment plan for user 400 to move toward target user profile vector 506 is determined. It will be appreciated that the method of FIG. 2 may be integrated into the method of FIG. 1 in the case that target user profile vector 506 was not previously determined. As this may be an iterative process, target user profile vector 506 may have already been determined in a previous iteration.

FIG. 3 is a flowchart of a method of selecting a treatment plan in accordance with an embodiment of the disclosed concept. The method of FIG. 3 may be applied in step 104 of FIG. 1. The method of FIG. 3 begins at 300 where user profile vector 500 and target user profile vector 506 are received. The method continues at 302, where the effect of a treatment on user profile vector 500 is determined. This effect may be, for example, the distance the treatment will move user profile vector 500 toward target user profile vector 506. At 304, the optimal treatment is selected. In an embodiment, the optimal treatment plan may be determined as the treatment plan that moves user profile vector 500 closest towards target user profile vector 506. It will be appreciated that selection of the optimal treatment may be determined using reinforcement machine learning such as a reinforcement learning based optimization approach where the problem of choosing the treatment is defined as a Markov Decision Process.

FIG. 5 provides a conceptual representation of moving from user profile vector 500 toward target user profile vector 506. In FIG. 5, there are two intermediate user profile vectors 502,504. In the first iteration, user 400 a first treatment plan causes user profile vector 500 to move to first intermediate user profile vector 502. From there, a second treatment plan causes first intermediate user profile vector 502 to move to second intermediate user profile vector 504. Finally, a third treatment plan causes second intermediate user profile vector 506 to move to target user profile vector 506. In each iteration, the user profile vector corresponding to user 400 is moved closer to target user profile vector 506. To determine the treatment plan at each iteration, reinforcement learning algorithms may be used.

As an example of determining an optimal treatment plan, assume the existence of a cost function C:f,S_{p,t},Gₚ→R (where C is the cost, f is treatment, S is the user profile vector for a user p at time t, G is the goal, and R is the reward), which can be used to rank a treatment's effectiveness. Also assume the existence of an optimal treatment for user p at a particular time t at state S_{p,t}. Let f*=fⱼ*(S_{p,t}) be the optimal treatment, with no guarantee as to the uniqueness of f*. Then in the procedure described above, at every time step, the treatment fⱼ that is chosen to be applied to the user is f*. Rephrased, at every time step, the treatment applies the most effective, i.e. highly ranked, treatment from the possible treatments.

The assumption of the cost function C:f,S_{p,t},Gₚ→R was made. While this function is not readily accessible, reinforcement learning may be applied to learn a variant of it. In particular, consider C′:f,P,Gₚ→R (where P is a persona-type). For a sufficient definition of closeness, if s₁ and s₂ (user profile vectors) are close to each other, then they belong to the same persona-type or cluster. Essentially, P is a set of states that are within some distance of one another. Thus, this function maps a <treatment f, persona-type P, goal G> tuple to a number representing the cost C′ of that treatment f for that persona-type P when attempting to achieve goal G. To determine the function C′, a reinforcement learning framework that uses a reward based feedback system is used.

In an embodiment, cluster analysis may be used to compare the user with a generic cluster profile that the user falls in. The generic cluster profile can be used to determine the target user vector profile for the user and the optimal treatment plan based on the success of treatments with other users in the cluster. Cluster analysis may be done on objective and subjective data from an anonymized database of some or all of the users of the system.

In an embodiment of the reinforcement learning system, each states s (i.e., user profile vector) is assigned a value Vₛ, and a possible set of actions A (treatments/challenges), along with a stochastic model for resulting states after applying a treatment from A to s. For example, in ∀s:

$${V}_{s} = {max}_{a} {\sum }_{{s}^{′}} P ( {s}^{′} \vert a , s ) * ( γ {V}_{{s}^{′}} + R ( {s}^{′} , s , a ) ) ,$$

the reward function is parameterized by the treatment applied, the starting position and the result position, and also the appropriate discount rate is taken into account. Next, a simple policy extraction can be applied to determine the optimal path for a user to reach the target user profile vector. While this is an example of a technique that may be used to determine a particular treatment plan, it will be appreciated that modifications may be made without departing from the scope of the disclosed concept.

In an embodiment, the optimal treatment plan may be based on several factors. For example and without limitation, the optimal treatment plan may be based on the user profile vector, the user's habits and behavior related information, a historic user response to the treatment in terms of health/benefit outcome (i.e., movement toward the target user profile vector), and subjective feedback (e.g., a preference) for the treatment. This information may be related to a user or a user cluster. All or a subset of these types of information may be used in deciding the optimal treatment. Additionally, the information may be given different weightings.

Referring back to FIG. 1, once an optimal treatment plan is determined at 104, the method proceeds to 106, where the treatment plan is provided to user 400. The treatment plan may be provided using any suitable means (e.g., without limitation, visual, audio, text, etc.). The treatment plan may include one or more tasks or goals for user 400 to pursue such as, without limitation, exercising a certain amount, reducing usage of electronic devices a certain amount and at certain times, particular recommendations for improving diet, etc.

After the treatment plan is provided to user 400 at 106, the method may return to 100 for another iteration. A period of time may pass between each iteration. The period may be fixed or fluid. For example, user 400 may initiate a second iteration after a defined period, such as a week, after meeting a defined treatment plan goal, at the user's own discretion, etc.

FIG. 6 is a schematic diagram of a system for developing and providing personalized treatment plans in accordance with an embodiment of the disclosed concept. The system of FIG. 6 is an example of a system that may be employed to implement the methods described with respect to FIGS. 1-3.

The system of FIG. 6 includes a user device 412. User device 412 may be an electronic device such as a smart phone or a computer that user 400 may interact with. User device 412 is also user to gather information, such as objective 402 and subjective 404 inputs association with user 400 and their goals. User device 412 may be connected to a backend system 800 via a communication means such as network 600. Other user device 700 may also be connected to backend system 800 via network 600.

Backend system 800 may be comprised of one or more devices such as computers or servers. Backend system 800 may include one or more processing units 802 and memories 804. Processing units 802 may be, for example and without limitation, a microprocessor, a microcontroller, or some other suitable processing device or circuitry, that interfaces with the memory. Memories 804 can be any of one or more of a variety of types of internal and/or external storage media such as, without limitation, RAM, ROM, EPROM(s), EEPROM(s), FLASH, and the like that provide a storage register, i.e., a machine readable medium, for data storage such as in the fashion of an internal storage area of a computer, and can be volatile memory or nonvolatile memory.

Backend system 800 may be configured to perform functions such as determining user profile vectors and determining optimal treatment plans for users. Backend system 800 may store various information from user 400 and other users and implement the machine learning algorithms used in determining user profile vectors and optimal treatment plans. As backend system 800 gathers more and more data over time, the machine learning algorithms will improve and more accurate user profile vectors and more optimal treatment plans may be determined. While backend system 800 is an example of a system that may implement the methods of FIGS. 1-3, one having ordinary skill in the art will understand that these methods may be implemented with other architectures without departing from the scope of the disclosed concept.

The disclosed concept described herein provides a data driven generation of user profile vectors that are semantically sound mathematical representations of users at various points in time. The disclosed concept also allows comparison of a user profile vector to user profile vectors of other users as well as giving reinforcement-learning based suggestions to the user to best complete coaching tasks. This data driven and machine learning based method and system can provide a digital platform for developing and providing treatment and/or coaching to a user desiring to achieve one or more goals. With the disclosed concept, the treatment and/or coaching are more effective than both existing digital platforms and personalized treatment or coaching. The disclosed concept provides more personalization than existing digital platforms and can draw on significantly more knowledge and data than a personal coach is capable of.

While examples of the disclosed concept have been described with respect to providing coaching and/or treatment to improve the sleep of a user, the disclosed concept may be applied to many fields. Additionally, while the disclosed concept is described with respect to providing coaching and/or treatment to a user, the disclosed concept is also relevant to other applications such as research or evaluation. For example, the disclosed concept may be used for research into the effectiveness of different types of treatments or understanding the association between characteristics and behaviors of users and various conditions such as sleep quality. Thus, the disclosed concept has wide ranging applications, as will be understood by those having ordinary skill in the art.

It will be appreciated that the disclosed concept may also be embodied on a non-transitory computer readable medium. For example, the disclosed concept may be embodied on a non-transitory computer readable medium having computer readable code embodied thereon which, when executed by a processor, may cause the processor to implement any of the methods shown and described herein.

Although the disclosed concept has been described in detail for the purpose of illustration based on what is currently considered to be the most practical and preferred embodiments, it is to be understood that such detail is solely for that purpose and that the disclosed concept is not limited to the disclosed embodiments, but, on the contrary, is intended to cover modifications and equivalent arrangements that are within the spirit and scope of the appended claims. For example, it is to be understood that the disclosed concept contemplates that, to the extent possible, one or more features of any embodiment can be combined with one or more features of any other embodiment.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

What is claimed is:

1. A method of providing personalized treatment, the method comprising:
gathering first information associated with a user;
determining a first user profile vector based on first information associated with the user;
determining a first treatment plan to move the user from the first user profile vector toward a target user profile vector; and
providing the first treatment plan to the user.

2. The method of claim 1, further comprising:
gathering second information associated with the user;
determining a second user profile vector based on the second information;
determining a second treatment plan to move the user from the second user profile vector toward the target user profile vector; and
providing the second treatment plan to the user,
wherein the first information is associated with a first point in time and the second information is associated with a second point in time after the first point in time.

3. The method of claim 1, further comprising:
determining a target user profile vector, wherein determining the target user profile vector includes:
receiving input from the user;
determining a goal of the user; and
determining the target user profile vector based on the goal of the user.

4. The method of claim 3, wherein the goal of the user is associated with sleep quality of the user.

5. The method of claim 1, wherein determining the first treatment plan includes:
receiving the first user profile vector and the target user profile vector;
determining for a plurality of treatment plans, the effect of each of the plurality treatment plans on the first user profile vector; and
selecting from the plurality of treatment plans the treatment plan that moves the first user profile vector the greatest distance toward the target user profile vector.

6. The method of claim 1, wherein determining the first treatment plan includes selecting the first treatment plan from among a plurality of treatment plans using reinforcement machine learning.

7. The method of claim 6, the effects of the plurality of treatment plans on other users is used in determining the first treatment plan.

8. The method of claim 1, wherein the first user profile vector is a mathematical representation of the user at a particular point in time and the target user profile vector is a mathematical representation of the user in a desired state.

9. The method of claim 1, wherein the first information includes information associated with sleep of the user.

10. The method of claim 9, wherein the first information includes one or more of information on demographics, emotional attributes, caffeine usage, exercise, stress levels, diet, and evening activity of the user.

11. The method of claim 1, wherein gathering the first information includes using natural language processing to gather the first information associated with the user.

12. The method of claim 1, wherein determining the first user profile vector includes using multimodal machine learning and knowledge mapping to fuse data from different input sources, and wherein the first user profile vector is a high dimensional numeric representation of the user.

13. The method of claim 1, wherein the first information includes multiple pieces of information, and wherein determining the first user profile vector includes providing weightings to the multiple pieces of information.

14. A system comprising:
a user device structured to gather first information associated with a user;
a backend system structured to receive the first information, to determine a first user profile vector based on first information associated with the user, to determine a first treatment plan to move the user from the first user profile vector toward a target user profile vector, and to provide the first treatment plan to the user device,
wherein the user device is structured to provide the first treatment plan to the user.

15. A non-transitory computer readable medium having computer readable code thereon which, when executed by a processor causes the processor to implement a method of providing personalized treatment, the method comprising:
gathering first information associated with a user;
determining a first user profile vector based on first information associated with the user;
determining a first treatment plan to move the user from the first user profile vector toward a target user profile vector; and
providing the first treatment plan to the user.
